(19) United States
(12) Patent Application Publication
(10) Pub. No.: US 2021/0236583 A1
(43) Pub. Date: Aug. 5, 2021

(54) Targeting the renin-angiotensin system for treatment of environment-and pathogen-induced lung injury

(71) Applicant: Yuntao WU, Manassas, VA (US)

(72) Inventor: Yuntao WU, Manassas, VA (US)

(21) Appl. No.: 17/163,947

(22) Filed: Feb. 1, 2021

Related U.S. Application Data
(60) Provisional application No. 62/969,274, filed on Feb. 3, 2020.

(51) Int. Cl. A61K 38/00; A61K 31/4422

(52) U.S. Cl. CPC A61K 38/005; A61K 31/4422

(57) ABSTRACT

The present disclosure relates to the design and method for targeting the renin-angiotensin system (RAS) for therapeutics of lung diseases, particularly environment- and pathogen-induced lung injury. Provided herein are design, methodology, compositions, and the like for such for restoring the regulatory balance the RAS system for the management of lung diseases.

### CROSS REFERENCE TO RELATED APPLICATION

The present application claims benefit of U.S. provisional application No. 62/969,274 filed Feb. 3, 2020, the contents of which are incorporated herein by reference in their entirety.

### FIELD

The present disclosure relates to targeting the renin-angiotensin system (RAS) for therapeutics of lung diseases, particularly environment- and pathogen-induced lung injury. Provided herein are design, methodology, compositions, and the like for such for restoring the regulatory balance the RAS system for the management of lung diseases.

### INTRODUCTION

In the lungs, activation of local pulmonary RAS can affect the pathogenesis of lung injury via multiple mechanisms, such as an increase in vascular permeability and alterations of alveolar epithelial cells (Kuba et al., 2006; Specks et al., 1990). Activation of pulmonary RAS involves renin, the initial enzyme of the RAS activation cascade (FIG. 1). Renin cleaves angiotensinogen, a globular protein, to generate angiotensin I (Ang I, a decapeptide hormone). The angiotensin-converting enzyme (ACE) then converts Ang I to angiotensin II (Ang II, an octapeptide hormone). Ang II exerts vasoactive effects through binding to its receptors, the angiotensin II type I (AT1) and type II (AT2) receptors.

Angiotensin-converting enzyme 2 (ACE2) is a homologue of ACE and plays a pivotal role in balancing responses initiated from ACE (Donoghue et al., 2000; Imai et al., 2010; Tipnis et al., 2000). ACE2 hydrolyses Ang I to generate Ang-(1-9). ACE2 also hydrolyses Ang II to generate Ang-(1-7), which binds to the G-protein coupled receptor MAS (Reudelhuber, 2005; Santos et al., 2003) to antagonize many of the Ang II-mediated effects. Overall, ACE2 functions as a counter-regulatory enzyme by decreasing local Ang II concentrations.

In the lungs, RAS activity, ACE, and Ang II are intrinsically high, and ACE2 activities are also highly elevated to regulate the balance of Ang II/Ang-(1-7) levels (Kuba et al., 2006; Specks et al., 1990). High levels of Ang II can lead to increases in vascular permeability and pulmonary oedema (Fyhrquist and Saijonmaa, 2008; Marshall, 2003; Marshall et al., 2004). In mouse models of acute respiratory distress syndrome, ACE2 knockout mice displayed more severe symptoms, while overexpression of ACE2 had some protective effects (Imai et al., 2005). As an example, in SARS coronavirus (SARS-CoV) infection of mice, ACE2 serves as a viral entry receptor to allow viral entry and replication in ACE2+ cells (Li et al., 2003). Both viral replication and the viral Spike protein alone have been shown to selectively reduce ACE2 but not ACE expression (Kuba et al., 2005). In addition, SARS-CoV also induces rapid downregulation of ACE2 from the cell surface (Glowacka et al., 2010; Wang et al., 2008) and the release of catalytically active ACE2 ectodomains (Haga et al., 2008; Jia et al., 2009; Lambert et al., 2005). These results suggest that the physiological balance between ACE/ACE2 and Ang II/Ang-(1-7) is likely disrupted by SARS-CoV viral infection. This virus-mediated effect is expected to have a pathogenic role in lung injury (Imai et al., 2008; Kuba et al., 2006; Yamamoto et al., 2006). Compensation of ACE2 and balancing ACE/ACE2 function can be used to alleviate virus-induced severe lung injury. In addition, SARS-CoV-2 (2019-nCoV) also uses ACE2 as the receptor for viral entry and replication in lung cells (Zhou et al., 2020), suggesting, to me, that the physiological balance between ACE/ACE2 and Ang II/Ang-(1-7) may also be similarly disrupted as SARS-CoV viral infection. Targeting ACE2 can block viral entry and reduce virus-replication induced lung pathogenesis.

### SUMMARY

Provided herein are several therapeutic approaches to block environment- and pathogen-induced lung injury. Such injury can be caused by environmental agents and infection by pathogens such as bacteria and viruses, including SARS-coronavirus (SARS-CoV), 2019-novel coronavirus (2019-nCov or SARS-CoV-2), and other disease causing animal and human coronaviruses (FIG. 1). SARS-CoV-2 infection can be inhibited by the recombinant human ACE2 protein (FIG. 2)

In one aspect, provided herein are design and methodology for increasing ACE2 level and decreasing SARS-CoV-2 (or 2019-nCoV) infection, through direct injection of recombinant ACE2 protein or segment of biologically active ACE2 (FIG. 2).

In another aspect, provided herein are design and methodology for compensating functional ACE2 deficiency by delivering therapeutic vectors expressing high levels of ACE2 activity directly into lung tissues. Such vectors can be integrating or non-integrating lenti- and retro-viral vector, AAV (Adeno-associated virus) vectors, and other gene therapy vectors that can deliver genes into lungs (FIG. 3).

In another aspect, provided herein are design and methodology for therapeutic Ang-(1-7) heptapeptide that can be delivered to activate its receptor MAS and to counteract the activities of Ang II.

### BRIEF DESCRIPTION OF THE DRAWING

FIG. 1: Potential therapeutics for environment- and virus-induced lung injury based on balancing the Renin-angiotensin system (RAS).

FIG. 2: The application of recombinant human ACE2 proteins to inhibit and treat SARS-CoV-2 infection.

FIG. 3: The application of a lentiviral vector to stably over-express ACE2 in human cells.

### DETAILED DESCRIPTION

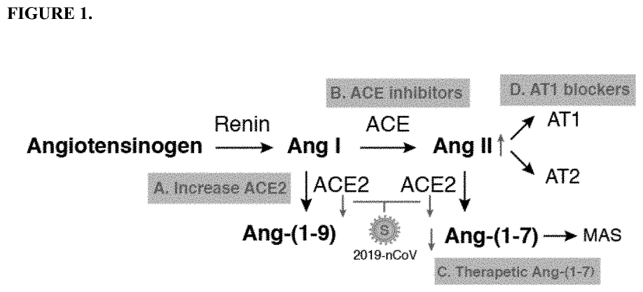

As shown in FIG. 1, Activation of the RAS cascade involves Renin and cleaves angiotensinogen to generate Ang I, which is then converted to Ang II by ACE. Ang II binds to its receptors (AT1 and AT2) to exert local and systemic effects such as vasoconstriction and promotion of the release of aldosterone. ACE2 functions as a counter-regulatory enzyme for balancing responses initiated from ACE. ACE2 hydrolyses Ang I and Ang II to generate Ang-(1-9) and Ang-(1-7). Ang-(1-7) binding to the MAS receptor antagonizes Ang II-mediated actions. As an example, viruses such as SARS-CoV and 2019-nCoV use ACE2 as the entry receptor. Inhibition of ACE2 expression or downregulation of surface ACE2 by these coronaviruses may disrupt function balances between ACE/ACE2, which may be alleviated by different approaches (FIG. 1. from A, B, C, to D).

There are several potential therapeutic approaches that can be tested or developed (FIG. 1). First, therapies to increase ACE2 expression can be developed, through direct injection of recombinant ACE2 protein, which has been shown to protect mice from severe acute lung injury (Imai et al., 2005), and by delivering therapeutic vectors expressing high levels of ACE2 directly into lung tissues to overcome virus-induced ACE2 deficiency. Second, certain ACE inhibitors such as lisinopril may be used to balance the ACE/ACE2 function. In addition, therapeutic Ang-(1-7) heptapeptide may be delivered to activate its receptor MAS and to counteract the activities of Ang II. Furthermore, drugs blocking Ang II receptors may also be tested. In particular, the type I, but not type II, Ang II receptor has been shown to promote disease pathogenesis by inducing lung oedemas and impairing lung function (Imai et al., 2005). Thus, a type I Ang II receptor blocker such as losartan could be tested for alleviating SARS-CoV-2-induced lung injury (FIG. 1).

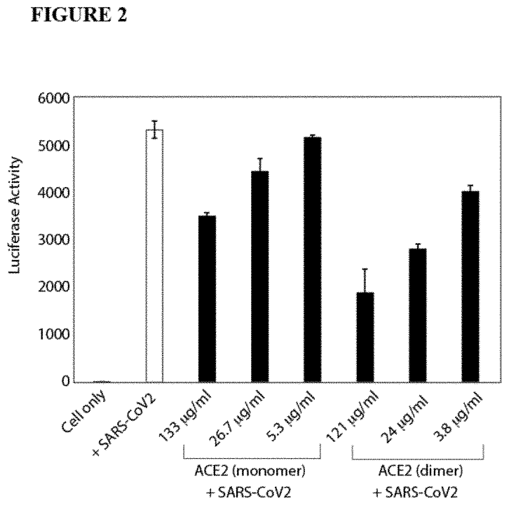

The use of recombinant human ACE2 proteins to block SARS-CoV-2 (or 2019-nCoV) virus infection of human cells. As shown in FIG. 2, HEK293T(ACE2/TMPRSS2) cells stably expressing human ACE2 and TMPRSS2 were used as the target cell for SARS-CoV-2 viral particle infection. The target cell was not infected (Cell only) or infected with a hybrid SARS-CoV-2 virus like particles (+SARS-CoV-2) carrying a luciferase reporter, as described previously (Hetrick et al., 2020). Target cells were also pre-treated with recombinant human ACE2 protein monomer (extracellular domain of ACE2, amino acid 1-615) or with ACE2 protein dimer (extracellular domain of ACE2, amino acid 1-720), and then infected in the presence of these ACE2 proteins in different concentrations (133 ug/ml to 5.3 ug/ml, ACE2 monomer; 121 ug/ml to 3.8 ug/ml, dimer). Following infection for 18 hours, viral entry and gene expression was quantified by luciferase assay. FIG. 2 shows ACE2 protein dosage-dependent inhibition of the infection of target cells by the hybrid SARS-CoV-2 particles.

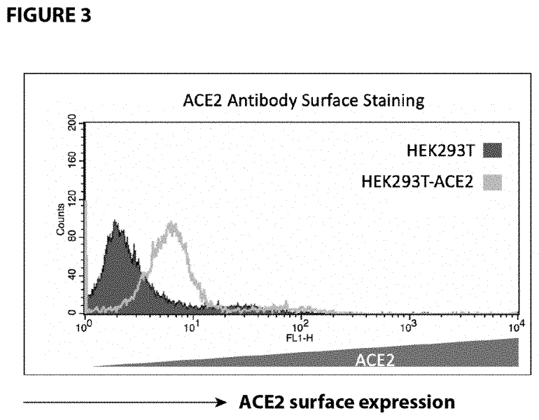

The use of a lentiviral vector to over-express ACE2 in human cells. As shown in FIG. 3, a lentiviral vector expressing the full-length human ACE2 gene (pLenti-hACE2-puro) was used to assemble a lentiviral particles, vLenti-hACE2-puro, which was then used to transduce human HEK293T cells. Following transduction, cells were selected in puromycin (1 μg/ml) for cells stably expressing ACE2; the puromycin gene was co-expressed with the ACE2 gene from pLenti-hACE2-puro. Cells that stably transduced with vLenti-hACE2-puro were analyzed for ACE2 over-expression by surface staining of ACE2 with a commercial anti-human ACE2 antibody and flow cytometry. Shown is the over-expression of ACE2 in cells stably transduced with vLenti-hACE2-puro (HEK293T-ACE2), in comparison with the un-transduced HEK293T cells (HEK293T).

Illustrative Examples are presented below. They are exemplary and non-limiting.

### Example 1: Potential Therapeutics for Environment- and Virus-Induced Lung Injury Based on Balancing the Renin-Angiotensin System (RAS)

As exemplified in FIG. 1, SARS-CoV-2-induced lung injury can be inhibited by balancing the RAS system.

As exemplified in FIG. 2, SARS-CoV-2 virus entry and infection of target cells can be blocked by the application of recombinant ACE2 proteins to inhibit viral infection of ACE2+ target cells, thus reducing virus-induced lung injury.

As exemplified in FIG. 3, the use of a lentiviral vector to over-express ACE2 to increase expression of ACE2 in cells.

### Experimental Procedures

Inhibition of Hybrid SARS-CoV-2 Reporter Virus Infection by Recombinant Human ACE2 Proteins

HEK293T(ACE2/TMPRSS2) cells were seeded into a 12-well tissue culture plate (2×10⁵ cells per well) in 1 ml cell culture medium (DMEM+10% heat-inactivated FBS, lx penicillin-streptomycin). Cell were grown overnight at 37° C.

Dilution of recombinant human ACE2 proteins

Dilution of purified recombinant human ACE2 monomeric protein (containing ACE2 extracellular domain, amino acid 1-615).

A—ACE2 monomer protein concentration, 2,000 μg/ml

A1—1:5 dilution—took 30 μl A+120 μl culture medium, concentration: 400 μg/ml

A2—1:25 dilution, took 30 μl A1+120 μl culture medium, concentration: 80 μg/ml

A3—1:125 dilution, took 30 μl A2+120 μl culture medium, concentration, 16 μg/ml

Dilution of purified recombinant human ACE2 dimeric protein (containing ACE2 extracellular domain, amino acid 1-720)

B—ACE2 dimer protein concentration, 1400 μg/ml

B1—1:5 dilution—took 42 μl B+120 μl culture medium, concentration: 363 μg/ml

B2—1:25 dilution, took 30 μl B1+120 μl culture medium, concentration: 72 μg/ml

B3—1:125 dilution, took 30 μl B2+120 ul culture medium, concentration, 14.5 μg/ml

100 μl of A1, A2, A3, B1, B2, or B3 were mixed with 100 μl of hybrid SARS-CoV-2(Luc) reporter virus, individually. As a control, 100 μl cell culture medium was mixed with 100 μl of hybrid SARS-CoV-2(Luc) reporter virus. Each resulting mixture was incubating at 37° C. for 30 minutes. Samples were labeled as A1+virus, A2+virus, A3+virus, B1+virus, B2+virus, B3+virus, and C+virus.

For infection of cells, 900 μl of culture medium were removed from each well of the 12-well plate, and 100 μl of medium were left in each well. The 200 μl of the mixture of A1 to B3 plus virus (prepared in step 3) were added. For control, the 200 μl of the mixture of medium plus the reporter virus were added. Cells were infected for 2 hours at 37° C. Infected cells were washed once, and then 1 ml fresh medium was added. The infected cells continued to culture for 18 hours.

Analysis of viral infection by luciferase assay. The infected cells were harvested and placed in a 1.5 ml micro-centrifuge tube. The cells were pelleted by centrifugation for 1 minute in a microfuge. The cell pellet was washed once with 1×cold PBS and then the cell pellet was resuspended in 100 μl Luciferase Assay Lysis Buffer. Luminescence was measured by using GloMax Discover Microplate Reader. The results are shown in FIG. 2.

The Use of Lentiviral Vector to Over-Express ACE2 in Human Cells

Assembly of lentiviral particles for ACE2 over-expression:

HEK293T cells were seeded into 10-cm tissue culture dish (3×10⁶ cells per dish) in 10 ml cell culture medium (DMEM+10% heat-inactivated Fetal Bovine Serum, lx penicillin-streptomycin). Cells were grown overnight at 37° C.

The next morning, cell culture medium was replaced with serum-free DMEM medium, 9 ml per dish.

The transfection mixture was prepared with 10 μg of pLenti-hACE2-puro vector, 8 μg of pCMVΔR8.2, and 2 μg of pHCMV-VSV-G. Serum-free DMEM medium was added to the DNA mixture to a total volume of 500 μl.

Transfectin™ (from Virongy LLC) was used for DNA transfection. 45 μl of Transfectin™ was added into 455 μl of Serum-free DMEM medium for a final volume of 500 μl. For transfection, 20 μg of DNA mixture (in 500 μl serum-free DMEM medium) prepared above was mixed with 500 μl Transfectin™ mixture. The combined solution was vigorously mixed and then added into HEK293T cells for 6 hours. The transfection supernatant was removed, and replaced with 10 fresh DMEM medium with 10% heat-inactivated Fetal Bovine Serum. Cells were cultured for 48 hours at 37° C.

The viral particles were harvested at 48 hours post cotransfection and were named as vLenti-hACE2-puro.

Transduction of HEK293T cells with vLenti-hACE2-puro for ACE2 over-expression:

HEK293T cells were cultured in 6-wells plate by seeding 2×10⁶ cells and grown overnight at 37° C.

500 μl of vLenti-hACE2-Puro viral particles were seeded for infection of cells for overnight.

2 ml fresh medium were added the next day and cultured for 2 more days.

Puromycin at 1 μg/ml were added. The cells were allowed to continue to culture, and the medium was changed every two days with fresh puromycin (1 μg/ml) being added into the medium.

The puromycin-resistant cells were transferred to T25 flasks, and then, to T75 flasks to grow cells to large volumes. Following selection, all cells were puromycin-resistant.

For surface staining analysis of ACE2 over-expression, both the parental HEK293T cells and the vLenti-hACE2-puro stably transduced HEK293T(ACE2) cells were used for staining with an anti-human ACE2 antibody. Cells were analyzed by flow cytometry as shown in FIG. 3. The vLenti-hACE2-puro transduced cells had higher ACE2 surface expression.

### REFERENCES

Donoghue, M., Hsieh, F., Baronas, E., Godbout, K., Gosselin, M., Stagliano, N., Donovan, M., Woolf, B., Robison, K., Jeyaseelan, R., et al. (2000). A novel angiotensin-converting enzyme-related carboxypeptidase (ACE2) converts angiotensin I to angiotensin 1-9. Circ Res 87, E1-9.
Fyhrquist, F., and Saijonmaa, O. (2008). Renin-angiotensin system revisited. J Intern Med 264, 224-236.
Glowacka, I., Bertram, S., Herzog, P., Pfefferle, S., Steffen, I., Muench, M. O., Simmons, G., Hofmann, H., Kuri, T., Weber, F., et al. (2010). Differential downregulation of ACE2 by the spike proteins of severe acute respiratory syndrome coronavirus and human coronavirus NL63. J Virol 84, 1198-1205.
Haga, S., Yamamoto, N., Nakai-Murakami, C., Osawa, Y., Tokunaga, K., Sata, T., Yamamoto, N., Sasazuki, T., and Ishizaka, Y. (2008). Modulation of TNF-alpha-converting enzyme by the spike protein of SARS-CoV and ACE2 induces TNF-alpha production and facilitates viral entry. Proc Natl Acad Sci USA 105, 7809-7814.
Hetrick, B., He, S., Chilin, L. D., Dabbagh, D., Alem, F., Narayanan, A., Luchini, A., Li, T., Liu, X., Liotta, L., et al. (2020). Development of a novel hybrid alphavirus-SARS-CoV-2 particle for rapid in vitro screening and quantification of neutralization antibodies, antiviral drugs, and viral mutations. bioRxiv, 2020.2012.2022.423965.
Imai, Y., Kuba, K., Ohto-Nakanishi, T., and Penninger, J. M. (2010). Angiotensin-converting enzyme 2 (ACE2) in disease pathogenesis. Circ J 74, 405-410.
Imai, Y., Kuba, K., and Penninger, J. M. (2008). The discovery of angiotensin-converting enzyme 2 and its role in acute lung injury in mice. Exp Physiol 93, 543-548.
Imai, Y., Kuba, K., Rao, S., Huan, Y., Guo, F., Guan, B., Yang, P., Sarao, R., Wada, T., Leong-Poi, H., et al. (2005). Angiotensin-converting enzyme 2 protects from severe acute lung failure. Nature 436, 112-116.
Jia, H. P., Look, D. C., Tan, P., Shi, L., Hickey, M., Gakhar, L., Chappell, M. C., Wohlford-Lenane, C., and McCray, P. B., Jr. (2009). Ectodomain shedding of angiotensin converting enzyme 2 in human airway epithelia. Am J Physiol Lung Cell Mol Physiol 297, L84-96.
Kuba, K., Imai, Y., and Penninger, J. M. (2006). Angiotensin-converting enzyme 2 in lung diseases. Curr Opin Pharmacol 6, 271-276.
Kuba, K., Imai, Y., Rao, S., Gao, H., Guo, F., Guan, B., Huan, Y., Yang, P., Zhang, Y., Deng, W., et al. (2005). A crucial role of angiotensin converting enzyme 2 (ACE2) in SARS coronavirus-induced lung injury. Nat Med 11, 875-879.
Lambert, D. W., Yarski, M., Warner, F. J., Thornhill, P., Parkin, E. T., Smith, A. I., Hooper, N. M., and Turner, A. J. (2005). Tumor necrosis factor-alpha convertase (ADAM17) mediates regulated ectodomain shedding of the severe-acute respiratory syndrome-coronavirus (SARS-CoV) receptor, angiotensin-converting enzyme-2 (ACE2). J Biol Chem 280, 30113-30119.
Li, W., Moore, M. J., Vasilieva, N., Sui, J., Wong, S. K., Berne, M. A., Somasundaran, M., Sullivan, J. L., Luzuriaga, K., Greenough, T. C., et al. (2003). Angiotensin-converting enzyme 2 is a functional receptor for the SARS coronavirus. Nature 426, 450-454.
Marshall, R. P. (2003). The pulmonary renin-angiotensin system. Curr Pharm Des 9, 715-722.
Marshall, R. P., Gohlke, P., Chambers, R. C., Howell, D. C., Bottoms, S. E., Unger, T., McAnulty, R. J., and Laurent, G. J. (2004). Angiotensin II and the fibroproliferative response to acute lung injury. Am J Physiol Lung Cell Mol Physiol 286, L156-164.
Reudelhuber, T. L. (2005). The renin-angiotensin system: peptides and enzymes beyond angiotensin II. Curr Opin Nephrol Hypertens 14, 155-159.
Santos, R. A., Simoes e Silva, A. C., Maric, C., Silva, D. M., Machado, R. P., de Buhr, I., Heringer-Walther, S., Pinheiro, S. V., Lopes, M. T., Bader, M., et al. (2003). Angiotensin-(1-7) is an endogenous ligand for the G protein-coupled receptor Mas. Proc Natl Acad Sci USA 100, 8258-8263.
Specks, U., Martin, W. J., 2nd, and Rohrbach, M. S. (1990). Bronchoalveolar lavage fluid angiotensin-converting enzyme in interstitial lung diseases. Am Rev Respir Dis 141, 117-123.
Tipnis, S. R., Hooper, N. M., Hyde, R., Karran, E., Christie, G., and Turner, A. J. (2000). A human homolog of angiotensin-converting enzyme. Cloning and functional expression as a captopril-insensitive carboxypeptidase. J Biol Chem 275, 33238-33243.
Wang, S., Guo, F., Liu, K., Wang, H., Rao, S., Yang, P., and Jiang, C. (2008). Endocytosis of the receptor-binding domain of SARS-CoV spike protein together with virus receptor ACE2. Virus Res 136, 8-15.
Yamamoto, K., Ohishi, M., Katsuya, T., Ito, N., Ikushima, M., Kaibe, M., Tatara, Y., Shiota, A., Sugano, S., Takeda, S., et al. (2006). Deletion of angiotensin-converting enzyme 2 accelerates pressure overload-induced cardiac dysfunction by increasing local angiotensin II. Hypertension 47, 718-726.
Zhou, P., Yang, X.-L., Wang, X.-G., Hu, B., Zhang, L., Zhang, W., Si, H.-R., Zhu, Y., Li, B., Huang, C.-L., et al. (2020). A pneumonia outbreak associated with a new coronavirus of probable bat origin. Nature 579, 270-273.

What is claimed is:

1. A method for treating environment- or pathogen-induced lung injury in a subject in need thereof by increasing Angiotensin-converting enzyme 2 (ACE2) expression, comprising injecting the subject with a recombinant ACE2 protein or segment of biologically active ACE2 protein.

2. The method of claim 1, wherein the subject is human.

3. The method of claim 2, wherein the subject is in need of treatment for SARS-coronavirus (SARS-Cov), SARS-CoV-2 (or 2019-novel coronavirus, 2019-nCov).

4. The method of claim 1, wherein the subject is in need of treatment for SARS-coronavirus (SARS-CoV), SARS-CoV-2 (or 2019-novel coronavirus, 2019-nCov).

5. A method for treating environment- or pathogen-induced lung injury in a subject in need thereof by compensating functional Angiotensin-converting enzyme 2 (ACE2) deficiency, comprising delivering to the subject one or more therapeutic vectors expressing ACE2 in lung tissue of the subject.

6. The method of claim 5, wherein the therapeutic vector is chosen from integrating or non-integrating lenti- and retro-viral vectors, AAV (Adeno-associated virus) vectors, and other gene therapy vectors that can be deliver genes into lungs.

7. The method of claim 6, wherein the subject is human.

8. The method of claim 7, wherein the subject is in need of treatment for SARS-coronavirus (SARS-CoV), SARS-CoV-2 (or 2019-novel coronavirus, 2019-nCov).

9. The method of claim 6, wherein the subject is in need of treatment for SARS-coronavirus (SARS-CoV), SARS-CoV-2 (or 2019-novel coronavirus, 2019-nCov).

10. The method of claim 5, wherein the subject is human.

11. The method of claim 10, wherein the subject is in need of treatment for SARS-coronavirus (SARS-CoV), SARS-CoV-2 (or 2019-novel coronavirus, 2019-nCov).

12. The method of claim 5, wherein the subject is in need of treatment for SARS-coronavirus (SARS-CoV), SARS-CoV-2 (or 2019-novel coronavirus, 2019-nCov).

13. A method for treating environment- or pathogen-induced lung injury in a subject in need thereof, comprising administering to the subject a composition comprising one or more active ingredients chosen from a therapeutic Ang-(1-7) heptapeptide, an ACE inhibitor, and a type I angiotensin II receptor blocker.

14. The method of claim 13, wherein the ACE inhibitor is lisinopril and wherein the type I angiotensin II receptor blocker is losartan.

15. The method of claim 14, wherein the subject is human.

16. The method of claim 15, wherein the subject is in need of treatment for SARS-coronavirus (SARS-CoV), SARS-CoV-2 (or 2019-novel coronavirus, 2019-nCov).

17. The method of claim 14, wherein the subject is in need of treatment for SARS-coronavirus (SARS-CoV), SARS-CoV-2 (or 2019-novel coronavirus, 2019-nCov).

18. The method of claim 13, wherein the subject is human.

19. The method of claim 18, wherein the subject is in need of treatment for SARS-coronavirus (SARS-CoV), SARS-CoV-2 (or 2019-novel coronavirus, 2019-nCov).

20. The method of claim 20, wherein the subject is human,
wherein the subject is in need of treatment for SARS-coronavirus (SARS-CoV), SARS-CoV-2 (or 2019-novel coronavirus, 2019-nCov).
wherein the ACE inhibitor is lisinopril, and
wherein the type I angiotensin II receptor blocker is losartan.
